(19) United States
(12) Patent Application Publication
(10) Pub. No.: US 2026/0062679 A1
(43) Pub. Date: Mar. 5, 2026

(54) HEPARINIZED MICROSPHERE, METHOD OF PREPARING THEREOF AND METHOD OF ISOLATING EXOSOMES

(71) Applicant: National Taiwan University of Science and Technology, TAIPEI CITY (TW)

(72) Inventors: Hsieh-Chih TSAI, New Taipei City (TW); Shuian-Yin LIN, Hsinchu County (TW); Kuan-Ju CHEN, Taipei City (TW)

(21) Appl. No.: 19/033,484

(22) Filed: Jan. 22, 2025

(30) Foreign Application Priority Data
Aug. 27, 2024 (TW) 113132200

(51) Int. Cl. C12N 5/09; C08J 3/16; C08J 3/24

(52) U.S. Cl. CPC C12N 5/0693; C08J 3/16; C08J 3/245; C08J 2305/12

(57) ABSTRACT

A heparinized microsphere is provided, including a polymer, heparin and a cross-linking agent. The polymer has at least one functional group includes a hydroxyl group, a carboxyl group, an amine group, or a combination thereof. The polymer and the heparin are cross-linked by a cross-linking agent. A method of preparing heparinized microsphere and a method of isolating exosomes are also provided to achieve purification of exosomes with high purity.

### CROSS-REFERENCE TO RELATED APPLICATION

This application claims priority to Taiwan Application Serial Number 113132200, filed on Aug. 27, 2024, which is herein incorporated by reference in its entirety.

### BACKGROUND

### Field of Invention

The present invention relates to a heparinized microsphere, method of preparing thereof and method of isolating exosomes.

### Description of Related Art

Exosomes are extracellular vesicles (EVs) secreted by cells outside the cells. Exosomes carrying various biological molecules such as proteins, nucleic acids, lipids, etc. play a crucial role in intercellular communication and the regulations of physiological and pathological processes. Therefore, it has been a growing interest in research and discussion on various types of extracellular vesicles. However, isolating and purifying exosomes from culture media or biological fluids of cultured cells is still a difficult task; common purification methods are differential centrifugation or size exclusion chromatography. Although the operation is convenient and the purification procedure is fast, the problems of low purity and difficulty in amplification and mass production have not been overcome. Immunoaffinity chromatography has the advantage of high purity.

Therefore, the related art really needs to be improved.

### SUMMARY

The invention provides a heparinized microsphere, comprising: a polymer having at least one functional group, the at least one functional group comprising a hydroxyl group, a carboxyl group, an amino group, or a combination thereof; heparin; and a cross-linking agent, the polymer and the heparin cross-linked by the cross-linking agent.

The present disclosure provides a method of preparing a heparinized microsphere, comprising: providing a polymer; and processing a mixing process with the polymer, heparin and a cross-linking agent, wherein when the polymer and the heparin are in contact with the cross-linking agent, cross-linking is performed to obtain the heparinized microsphere.

The present disclosure also provides a method of isolating exosomes, comprising: mixing a solution containing a plurality of exosomes and a plurality of heparinized microspheres as claimed in claim 1 for combining the plurality of exosomes and the plurality of heparinized microspheres to obtain a plurality of exosome microspheres; and mixing a salt solution with the plurality of exosome microspheres to isolate the exosomes from the plurality of heparinized microspheres.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 depicts a schematic view of epoxide and cross-linked agarose microspheres (EpCAMs) with surface-modified epoxy groups according to some embodiments of the present disclosure.

FIG. 2 depicts a schematic view of EpCAMs bonded with heparin according to some embodiments of the present disclosure.

FIG. 3 shows an attenuated total reflection-Fourier transform infrared (ATR-FTIR) spectrum of microspheres according to some embodiments of the present disclosure.

FIG. 4 shows Raman spectroscopy images of microspheres according to some embodiments of the present disclosure.

FIG. 5 shows an effect of different epichlorohydrin (ECH) concentrations on epoxy group content in some embodiments of the present disclosure.

FIG. 6 shows an attenuated total reflection Fourier transform infrared spectrum of EpCAMs bonded with heparin according to some embodiments of the present disclosure.

FIG. 7 shows the Raman spectrum of EpCAMs bonded with heparin according to some embodiments of the present disclosure.

FIGS. 8A to 8F show particle size distribution diagrams (FIG. 8A) pre-purification using size exclusion or affinity capture methods according to some embodiments of the present disclosure; (FIG. 8B) size exclusion chromatography (SEC); affinity capture method (FIG. 8C) EpCAMs, (FIG. 8D) HepCAMs 0.25, (FIG. 8E) HepCAMs 0.50, (FIG. 8F) HepCAMs 1.00.

FIG. 9 shows transmission electron microscope (TEM) images of different purification methods of exosomes according to some embodiments of the present disclosure.

FIG. 10 shows immune characteristics of extracellular vesicles (CD9, CD63 and CD81) using different methods for purifying exosomes according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number. Still further, the present disclosure may repeat reference numerals and/or letters in the various examples. This repetition is for the purpose of simplicity and clarity and does not in itself dictate a relationship between the various embodiments and/or configurations discussed.

The potential application value of exosomes is very broad, so the isolation, purification and concentration of exosomes are crucial to evaluate their biological functions and their downstream applications. It is a great challenge in exosomes isolation because of their small size (about 30-150 nanometers (nm)), low density, and many similar molecular structures, such as cell debris, protein aggregates, and lipoproteins. Furthermore, the bioactivity and integrity of exosomes are susceptible to different isolation techniques. Therefore, how to effectively isolate and concentrate exosomes has become a major challenge in academic research and clinical application.

Some embodiments of the present disclosure provide a heparinized microsphere, including a polymer, heparin, and a cross-linking agent. The polymer has at least one functional group including a hydroxyl group, a carboxyl group, an amino group, or a combination thereof. The polymer and the heparin are crosslinked by a cross-linking agent.

In some examples, polymer includes agarose (having OH group), chitosan (having NH₂ group), hyaluronic acid (having OH group and COOH group), pectin (having OH group and COOH group), carboxymethyl cellulose (having OH group and COOH group), hydroxypropyl methylcellulose (having OH group), alginate (having OH group and COOH group), or a combination thereof.

As used herein, “agarose” refers to a neutral polysaccharide, generally extracted from red algae in the ocean. Agarose is a long chain polymer composed of dextrose galactose (D-galactose) and 3,6-anhydro-L-galactose. At 90-100° C., the hydrogen bonds between agarose structural units are broken. Agarose is dispersed in water in the form of irregular curls, forming a clear solution.

In some examples, the polymer comprises a physical cross-linked polymer, a chemical cross-linked polymer, or a combination thereof.

In some examples, cross-linking agent comprises epichlorohydrin (ECH).

In some examples, a content of an epoxy group at a surface of the polymer ranges from 200 μmol/g of the polymer to 600 μmol/g of the polymer. In some examples, the content of the epoxy group at the surface of the polymer is 200, 210, 220, 230, 240, 250, 270, 290, 300, 320, 340, 360, 380, 400, 420, 440, 460, 480, 500, 520, 540, 550, 560, 570, 580, 590, or 600 μmol/g of the polymer, or any value between any two of these values.

In some examples, a content of the heparin at a surface of the polymer ranges from 10 μg/mg of the polymer to 90 μg/mg of the polymer. In some examples, a content of the heparin at a surface of the polymer is 10, 15, 20, 25, 30, 35, 40, 41, 42, 43, 44, 45, 50, 55, 60, 61, 62, 63, 64, 65, 70, 75, 76, 77, 78, 79, 80, 85, or 90 μg/mg of the polymer, or any value between any two of these values.

In some examples, two ends of the cross-linking agent are respectively bonded to the at least one functional group of the polymer and at least one hydroxyl group of the heparin.

As used herein, “microsphere” refers to represent approximately spherical particles in the size range defined from 1 micrometer (μm) to 1000 μm.

Other embodiments of the present disclosure provide a method of preparing a heparinized microsphere, comprising: providing a polymer; and processing a mixing process with the polymer, heparin and a cross-linking agent, in which when the polymer and the heparin are in contact with the cross-linking agent, cross-linking is performed to obtain the heparinized microsphere.

In some examples, the providing the polymer comprises heating and dissolving a plurality of monomers in water to obtain an aqueous phase mixture. In some examples, the plurality of monomers are dissolved in water at a concentration of from 5 wt % to 25 wt %, such as 5%, 6%, 7%, 8%, 9%, 10%, 11%, 12%, 13%, 14%, 15%, 16%, 17%, 18%, 19%, 20%, 21%, 22%, 23%, 24%, 25%, or any value between any two of these values. If the concentration is too high or too low, the polymer sphere may become unstable, disintegrate, or fragment. In some examples, the plurality of monomers include, but are not limited to agarose, chitosan, hyaluronic acid, pectin, carboxymethylcellulose, hydroxypropyl methylcellulose, alginate, or a combination thereof. In some examples, heating temperature is from 50° C. to 120° C., such as 50° C., 60° C., 70° C., 80° C., 90° C., 100° C., 110° C., or 120° C., or any value between any two of these values.

In some examples, the providing the polymer further comprises mixing an oil and a surfactant to obtain an oil phase mixture. In some examples, the oil comprises alkanes, esters, or a combination thereof. In some examples, the oil includes alkanes with a boiling point above 100° C. The alkanes include, but are not limited to petroleum ether, paraffin oil, mineral oil, stearic acid, cottonseed oil, oleyl alcohol, white wax oil or a combination thereof. In some examples, the surfactant includes, but is not limited to sorbitan monooleate (span 80), hydroxylated lanolin, polyoxythylene sorbitol beeswax derivative, propylene glycol fatty acid ester, propylene glycol monolaurate, di(ethylene glycol) monooleate, sodium lauryl ether sulfate (2EO), polyoxythylene sorbitol beeswax derivative, diethylene glycol distearate, or a combination thereof.

In some examples, the step of providing the polymer further includes mixing the aqueous phase mixture and the oil phase mixture to obtain a water-in-oil (W/O) emulsion. In some examples, the water phase mixture and the oil phase mixture are mixed and heated at the same time. The heating temperature is from 40° C. to 70° C., such as 40° C., 45° C., 50° C., 55° C., 60° C., 65° C., 70° C., or any value between any two of these values. In some examples, the water-in-oil emulsion has an oil:water volume ratio ranging from about 10:1 to about 1:1, such as 10:1, 8:1, 7:1, 6:1, 5:1, 4:1, 3:1, 2:1, 1:1, or any value between any two of these values. In some examples, the water phase mixture and the oil phase mixture are mixed and stirred and rotated at a certain rotation speed from 300 rpm and 1500 rpm, such as 300 rpm, 400 rpm, 500 rpm, 600 rpm, 700 rpm, 800 rpm, 900 rpm, 1000 rpm, 1100 rpm, 1200 rpm, 1300 rpm, 1400 rpm, or 1500 rpm, or any value between any two of these values.

In some examples, the water-in-oil emulsion is cooled until set to obtain the polymer, such as physical cross-linked microsphere.

In some examples, the step of providing the polymer further includes cross-linking the microspheres and a cross-linking agent in an alkaline environment to obtain the polymer (for example, chemical cross-linked polymer). In some examples, the processing the mixing process with the polymer, heparin and the cross-linking agent comprises: cross-linking the cross-linking agent and the polymer at a volume ratio of from 0.01:1 to 1:1 in an alkaline solution to obtain a chemical cross-linked polymer and resulted swelling ratio (w/w) from 20 to 4; and mixing the chemical cross-linked polymer, the heparin and the cross-linking agent. In some examples, Epichlorohydrin (ECH) and physical cross-linked polymer are mixed in an alkaline solution at a ratio of from 0.01:1 to 1:1 (v/v) and resulted swelling ratio (w/w) from 20 to 4. In some examples, the ratio of Epichlorohydrin (ECH) to physical cross-linked polymer is, for example, 0.01:1 v/v, 0.05:1 v/v, 0.10:1 v/v, 0.15:1 v/v, 0.20:1 v/v, 0.25:1 v/v, 0.30:1 v/v, 0.35:1 v/v, 0.40:1 v/v, 0.45:1 v/v, 0.50:1 v/v, 0.55:1 v/v, 0.60:1 v/v, 0.65:1 v/v, 0.70:1 v/v, 0.75:1 v/v, 0.80:1 v/v, 0.85:1 v/v, 0.90:1 v/v, 0.95:1 v/v, 1:1 v/v, or any value between any two of these values. In some examples, swelling ratio is, for example, 20 w/w, 19 w/w, 18 w/w, 17 w/w, 16 w/w, 15 w/w, 14 w/w, 13 w/w, 12 w/w, 11 w/w, 10 w/w, 9 w/w, 8 w/w, 7 w/w, 6 w/w, 5 w/w, 4 w/w, or any value between any two of these values. In some examples, the alkaline solution includes, but is not limited to sodium hydroxide solution.

In some examples, the step of providing the polymer further comprises cross-linking the physical cross-linked polymer and the cross-linking agent in a neutral or acidic environment to obtain the polymer (such as, chemical cross-linked polymer). A chemical cross-linked polymer can also be obtained by matching the corresponding cross-linking agent for cross-linking in a neutral or acidic environment according to material and functional group of the microspheres.

In some examples, the step of processing a mixing process with the polymer, the heparin and the cross-linking agent, when the polymer and the heparin are in contact with the cross-linking agent, cross-linking is performed to obtain the heparinized microsphere. In some examples, the mixing process includes microfluidics, titration, electrospinning, emulsion polymerization, film emulsification, or a combination thereof.

In some examples, the polymer and the cross-linking agent are mixed in an alkaline solution at a volume ratio of from 0.001:1 to 0.15:1.

In some examples, the chemical cross-linked polymer and the cross-linking agent are cross-linked in an alkaline environment to obtain an epoxidized microsphere. Epichlorohydrin (ECH) and the chemical cross-linked polymer are mixed in alkaline solution at a volume ratio of from 0.001:1 to 0.15:1. In some examples, the volume ratio of the epichlorohydrin and the chemical cross-linked polymer is, such as, 0.001:1, 0.005:1, 0.010:1, 0.012:1, 0.014:1, 0.016:1, 0.018:1, 0.01:1, 0.02:1, 0.03:1, 0.04:1, 0.05:1, 0.06:1, 0.07:1, 0.08:1, 0.09:1, 0.10:1, 0.11:1, 0.12:1, 0.13:1, 0.14:1, or 0.15:1, or any value between any two of these values. In some examples, the alkaline solution includes, but is not limited to sodium hydroxide solution. The hydroxyl group (OH) of the polymer monomer reacts and bonds with epichlorohydrin to form an intermediate product through an ether bond. After the hydroxyl group on the original C₁ is deprotonated in an alkaline environment, it is easy to attack C₂ containing chlorine. Chlorine is a good leaving group, thereby obtaining an epoxy group, which is convenient for subsequent modification of heparin.

In some examples, a weight ratio of the polymer to the heparin is from 1:0.1 to 1:2.0.

In some examples, the epoxidized microsphere is bonded with the heparin to obtain the heparinized microsphere. The epoxidized microsphere and the heparin sodium salt are mixed and reacted in an acidic solution at a weight ratio of from 1:0.1 to 1:2.0. In some examples, a weight ratio of the epoxidized microsphere to the heparin is, such as, 1:0.1, 1:0.2, 1:0.3, 1:0.4, 1:0.5, 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, or 1:2.0, or any value between any two of these values. In some examples, the acidic solution includes, but is not limited to, hydrochloric acid solutions.

As used herein, “emulsification” refers to the dispersion of two or more mutually incompatible liquids. One liquid (dispersed phase) exists in the form of droplets and is dispersed in another liquid (continuous phase), and this phenomenon is called emulsification. The emulsion stability can be increased by adding surfactants or emulsifiers to prevent droplet aggregation. The types of emulsification can be divided into water-in-oil (W/O), oil-in-water (O/W) and multiple emulsification methods. If the dispersed phase is water and the continuous phase is oil, it is called a W/O emulsion; and if the dispersed phase is oil and the continuous phase is water, it is called an oil-in-water O/W emulsion.

Another embodiment of the present disclosure provides a method of isolating exosomes, comprising: mixing a solution containing a plurality of exosomes and a plurality of heparinized microspheres as above mentioned for combining the plurality of exosomes and the plurality of heparinized microspheres to obtain a plurality of exosome microspheres; and mixing a salt solution with the plurality of exosome microspheres to isolate the plurality of exosomes from the plurality of heparinized microspheres.

In some examples, after the mixing the salt solution with the plurality of exosome microspheres, the method further comprises mixing the plurality of exosome microspheres and a buffer to obtain a mixed buffer solution; and centrifuging the mixed buffer solution to obtain a supernatant containing the plurality of exosomes.

In some examples, a salt in the salt solution includes sodium chloride, phosphate, or a combination thereof.

In some examples, a concentration of the salt solution is from 1.5 M to 3 M.

As used herein, “extracellular vesicles” (EVs) refer to vesicles with an outer membrane composed of a bilayer of lipids and released from inside the cell to the extracellular space. EVs are released by all cells (including prokaryotes and eukaryotes). EVs are composed of multiple subtypes, including exosomes, microvesicles (MVs), and apoptotic bodies.

As used herein, “exosome” refers to that the formation process of exosomes is relatively complex. The cell membrane dents inward to form early endosomes. The early endosomes contain intracellular bioinformation molecules and further mature to form multivesicular body (MVBs). MVBs which secreted through exocytosis is call exosome.

Exosomes are rich in proteins, such as tetraspanins (CD9, CD63 and CD81), endosomal sorting complex required for transport (ESCRT) proteins (Alix, TSG101), integration proteins, heat shock proteins (Hsp), actin, among which CD9, CD63, CD81, Alix and Tsg101 are commonly used as exosome markers.

A number of examples are provided herein to elaborate the heparinized microspheres of the instant disclosure. However, the examples are for demonstration purpose alone, and the instant disclosure is not limited thereto.

### Example

For the sake of clarity, features and elements that are well known in the art and are not necessary for an understanding of the principles described have been omitted.

### Preparation Example 1 Preparation of Agarose Microspheres

First, an aqueous phase (dispersed phase) mixed solution and an oil phase (continuous phase) mixed solution were provided. In the aqueous phase mixture, an agarose powder (a plurality of monomers) was dissolved in water at a concentration of 5 wt % to 25 wt %, for example, 6 wt %. The agarose powder and deionized water were evenly mixed and then heated to completely dissolve the agarose powder in the deionized water to obtain an aqueous phase mixture. Then, in the oil phase, petroleum ether and paraffin oil were provided and mixed in a volume ratio of 2:3 as an oil phase liquid, and an emulsifier was added into the oil phase liquid and mixed evenly to obtain an oil phase mixture.

Next, the aqueous phase mixture and the oil phase mixture were mixed. The oil phase mixture was heated, and the aqueous phase mixture was slowly dropped into the oil phase mixture and continued to stir and rotate at a certain rotation speed to obtain a water-in-oil (W/O) emulsion. The water-in-oil emulsion was cooled so that the agarose in the aqueous phase mixture was shaped through physical cross-linking to obtain a polymer (i.e. microspheres). After the physical cross-linking reaction was completed, the oil phase was removed and washed, and the microspheres were dried to obtain physically cross-linked agarose microspheres (AMs). The emulsification method has the advantages of good dispersion and easy mass production.

Next, the physically cross-linked agarose microspheres were chemically cross-linked with a cross-linking agent. After dissolving epichlorohydrin (ECH) in acetone, ECH and the physically cross-linked agarose microspheres were added into a sodium hydroxide solution. After removing unreacted ECH and washing several times, the pH value was balanced to neutral to obtain chemical cross-linked agarose microspheres (CAMs).

### Preparation Example 2 Agarose Microspheres Surface Modified with Epoxy Group

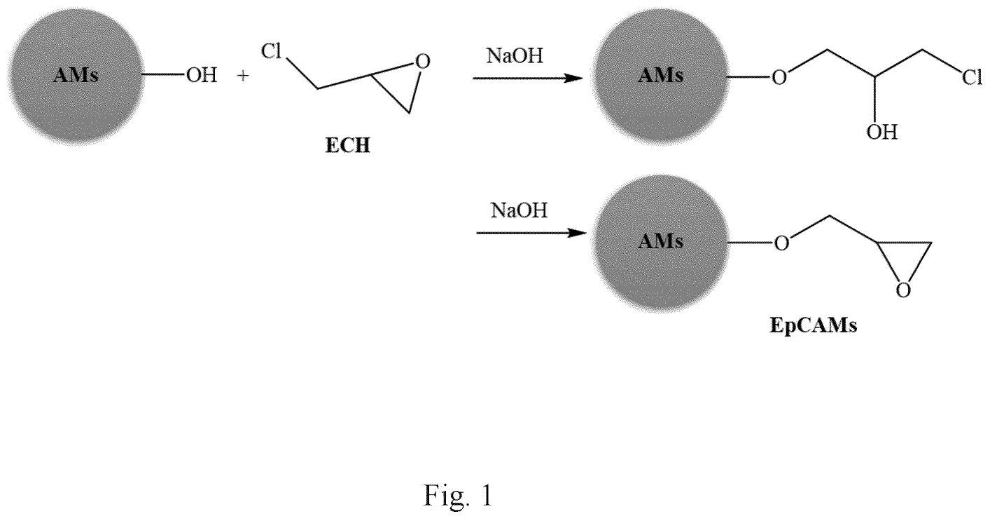

The chemical cross-linked agarose microspheres (CAMs) of Preparation Example 1 were reacted with a cross-linking agent. The epichlorohydrin (ECH) and CAMs in sodium hydroxide aqueous solution were mixed at a ratio of 0.014˜0.14 (v/v). After removing unreacted ECH and washing several times, the pH value was balanced to neutral, and the epoxide and cross-linked agarose microspheres (EpCAMs) with surface-modified epoxy groups were obtained. The synthesis schematic view is shown in FIG. 1.

### Preparation Example 3 EpCAMs Bonded with Heparin

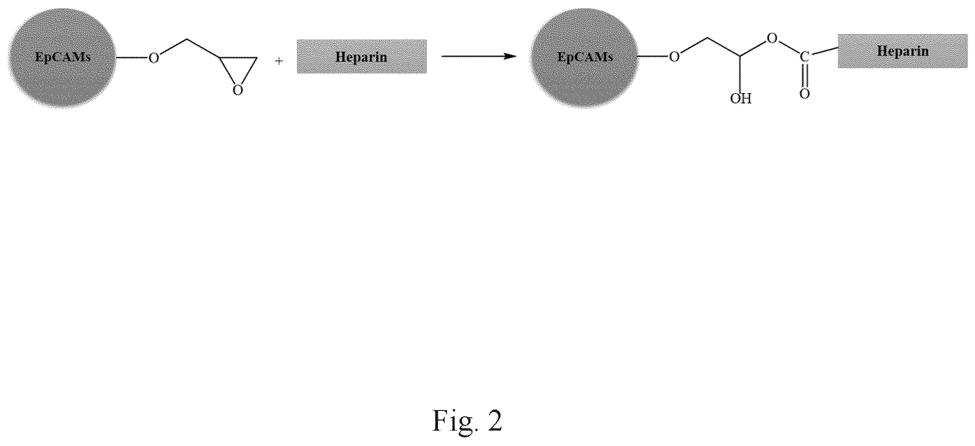

EpCAMs from Preparation Example 2 were added to the hydrochloric acid solution (pH=4), and the EpCAMs and sodium heparin were reacted at a ratio of from 0.1˜1.5 (v/v). Unreacted sodium heparin was removed and washed several times to balance the pH to neutral and dry, epoxidized agarose microspheres bonded with heparin (heparinized CAM, HepCAMs) were obtained. The synthesis schematic view is shown in FIG. 2.

### Example 1 Particle Size Analysis

The particle size analyzer used in the present disclosure measured the particle size of particle samples through laser diffraction technology. The present disclosure used the particle size analyzer to confirm the impact of the mechanical and stirring rotation speed on the particle size of agarose microspheres prepared by the emulsification method, and the rotation speed conditions were selected for the appropriate particle size required for subsequent experiments. The mechanical and stirring rotation speeds were 500 rpm, 750 rpm and 1000 rpm for granulation. As the rotation speed increases, the shear force on the dispersed phase increases, which causes the particles to be divided into smaller particle sizes.

After rehydrating the AMs and the CAMs in Preparation Example 1, the particle size was measured at a rotation speed of 1500 rpm. Span=(D₉₀−D₁₀)/D₅₀, in which Span is also called the span value, which infers the degree of uniformity of particle size; D₁₀ is the volume distribution of 10% particles; D₅₀ is the volume distribution of 50% particles; D₉₀ is the volume distribution of 90% particles.

TABLE 1

| Rotation | Microsphere | | | |
|---|---|---|---|---|
| speed (rpm) | type | D₁₀ (μm) | D₅₀ (μm) | D₉₀ (μm) |
| 500 | AMs | 210 | 367 | 593 |
| | CAMs | 145 | 223 | 338 |
| 750 | AMs | 133 | 215 | 328 |
| | CAMs | 82 | 155 | 284 |
| 1000 | AMs | 66 | 147 | 292 |
| | CAMs | 55 | 116 | 242 |

As shown in Table 1 above, the particle size of chemical cross-linked agarose microspheres (CAMs) was smaller than the particle size of physically cross-linked agarose microspheres (AMs). After chemically cross-linking, the agarose molecular chains became more compact, free-volume is compressed, and the volume of the particles shrinks. Among the three different rotation speed conditions, the particle size distributions of chemical cross-linked agarose microspheres (CAMs) prepared at 1000 rpm were D₁₀ 55 μm, D₅₀ 116 μm and D₉₀ 242 μm, which were the same as those of commercially available microspheres (45-150 μm). Therefore, the chemical cross-linked agarose microspheres (CAMs) prepared at the rotation speed of 1000 rpm were used in the present disclosure for subsequent experimental development and use.

### Example 2 Surface Morphology Analysis

In order to confirm the impact of physical cross-linking and chemical cross-linking on the surface morphology of agarose microspheres, a scanning electron microscope (SEM) was used in the present disclosure to observe dry agarose microspheres (the AMs and the CAMs in Preparation Example 1), and an optical microscope (OM) was used for observing the swollen agarose microspheres (rehydrate the AMs and the CAMs in Preparation Example 1).

It can be found that the dried physical cross-linked agarose microspheres (AMs) did not maintain in spherical shape, while the dried CAMs were completely in spherical shape (figure not shown). Therefore, the mechanical strength of CAMs was significantly improved and can still maintain spherical shape after drying. In addition, both AMs and CAMs can maintain a spherical shape in the swollen state, and their particle size was consistent with the particle size analyzer results (figure not shown).

Swelling Ratio with Crosslinking Agents

Agarose microspheres absorb a significant amount of water. Physically cross-linked agarose microspheres (AMs) can absorb up to 20 times their dry weight in water and typically contain 95% water within their structure. However, after chemical cross-linking, the swelling ratio and water content of the agarose microspheres decrease. This is because chemical cross-linking creates a denser network, reducing free space and increasing the proportion of agarose within the microspheres.

Epichlorohydrin (ECH) and physical cross-linked polymer (CAMs) are mixed in an alkaline solution at a ratio of 0.01, 0.5, and 1.0 (v/v), the swelling ratios are 19.6, 11.6, and 4.0, respectively (Table 1.1). These microspheres only absorb up to 5 times their dry weight in water, with a water content of approximately 85%. Epoxy modification or heparin conjugation does not significantly affect the internal structure of the microspheres and has minimal impact on their swelling ratio or water content.

TABLE 1.1

| Microsphere | Swelling ratio (w/w) |
|---|---|
| AMs | 22.1 |
| CAM (0.01) | 19.6 |
| CAM (0.5) | 11.6 |
| CAM (1.0) | 4 |

### Example 3 Agarose Microspheres Surface Modified with Epoxy Group

### (1) ATR-FTIR Spectral Analysis

In order to confirm whether the surface modification of epoxy groups on agarose microspheres was successful in the present disclosure, ATR-FTIR was used to observe changes in the functional group of the agarose powder (pure agarose), the CAMs of Preparation Example 1, and EpCAMs of Preparation Example 2.

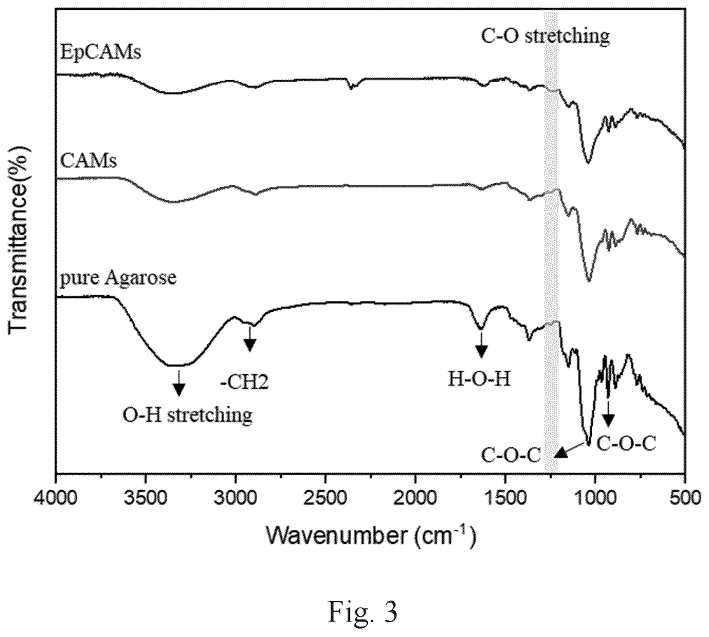

Please refer to FIG. 3. The characteristic peaks of original agarose powder (pure agarose) were O—H stretching vibration at 3350 cm⁻¹, —CH₂ at 2888 cm⁻¹, H—O—H at 1634 cm⁻¹ with stretching vibration of water molecules combined with polysaccharide, C—O—C antisymmetric stretching vibration of the glycosidic bond at 1038 cm⁻¹, and C—O—C vibration of the 3,6-anhydrogalactose unit at 930 cm⁻¹. CAMs did not produce different functional groups, so their characteristic peak positions overlapped with agarose powder. The absorption band of EpCAMs at 1237 cm⁻¹ was the characteristic peak of the epoxy group. This proves that the epoxy functional groups were successfully modified on the surface of CAMs in the present disclosure.

### (2) Raman Spectroscopy Analysis

In order to confirm whether the surface modification of epoxy groups on agarose microspheres is successful, the present disclosure uses Raman spectroscopy to observe changes in the functional groups and chemical bonds of agarose powder (pure agarose), the CAMs of Preparation Example 1 and the EpCAMs of Preparation Example 2.

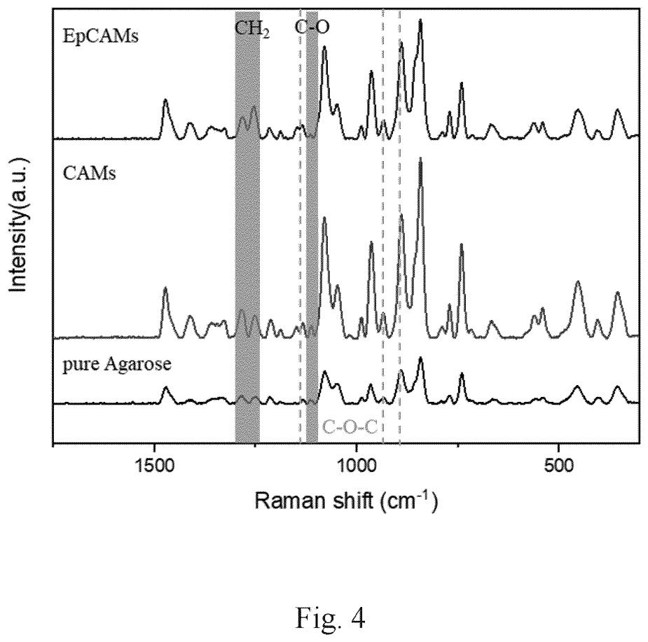

Please refer to FIG. 4, the original agarose powder (pure agarose) had the characteristic peaks of the C—O—C vibration of the glycosidic bond at the positions of 889 cm⁻¹, 934 cm⁻¹ and 1189 cm⁻¹. The CAMs did not produce different functional groups, so their characteristic peak positions overlapped with agarose powder (pure agarose). A slight difference in the positions of the characteristic peaks of EpCAMs and CAMs at 1249 cm⁻¹ and 1282 cm⁻¹ were CH2 vibrations. As for the position of 1112 cm⁻¹, the decrease in the intensity of the characteristic peak of EpCAMs was due to the C—O stretching vibration.

### (3) Analysis of Epoxy Group Content on the Surface of Agarose Microspheres

The reaction between oxirane ring and sodium thiosulphate (Na₂S₂O₄) will release hydroxide ions (OH), and then use hydrogen ions (H⁺) to adjust the solution to neutrality, and then measure the epoxy group content. According to Preparation Example 2, the addition amount of ECH is respectively 0.014, 0.07 and 0.14 v/v of agarose microspheres for epoxidation reaction, and the epoxy equivalent was tested. The calculation formula of epoxy equivalent is S=C_{HCl}(V0−V1)/m, S: epoxy equivalent (μmol/g), CHCl: hydrochloric acid concentration (mol/L), V0: volume before hydrochloric acid titration (mL), V1: volume after hydrochloric acid titration (mL), m: sample weight of surface modified epoxy groups (g).

Please refer to FIG. 5, the epoxy group content increased with the increase of ECH concentration. However, it can be found that when the ECH concentration increased from 0.07 (v/v) to 0.14 (v/v), the increase curve of epoxy group content tends to be flat, and it is estimated that the surface energy modified epoxy group tends to be saturated. The effect of different ECH concentrations (0.014, 0.07 and 0.14 (v/v)) on the epoxy group content can be more clearly observed from Table 2. In the present disclosure, EpCAMs prepared with the ECH concentration of 0.07 (v/v) were selected for use in subsequent examples.

TABLE 2

| | ECH/0.5M | Epoxy group density |
|---|---|---|
| | NaOH (v/v) | (μmol/g of agarose) |
| CAMs | 0.000 | 3.95 |
| EpCAMs | 0.014 | 216.65 |
| | 0.070 | 550.55 |
| | 0.140 | 578.25 |

### Example 4 EpCAMs Bonded with Heparin

### (1) ATR-FTIR Spectral Analysis

In order to confirm whether the surface modification of heparin on epoxidized agarose microspheres was successful in the present disclosure, ATR-FTIR was used to observe changes in the functional group of the agarose powder (pure agarose), the EpCAMs of Preparation Example 3, and HepCAMs of Preparation Example 3.

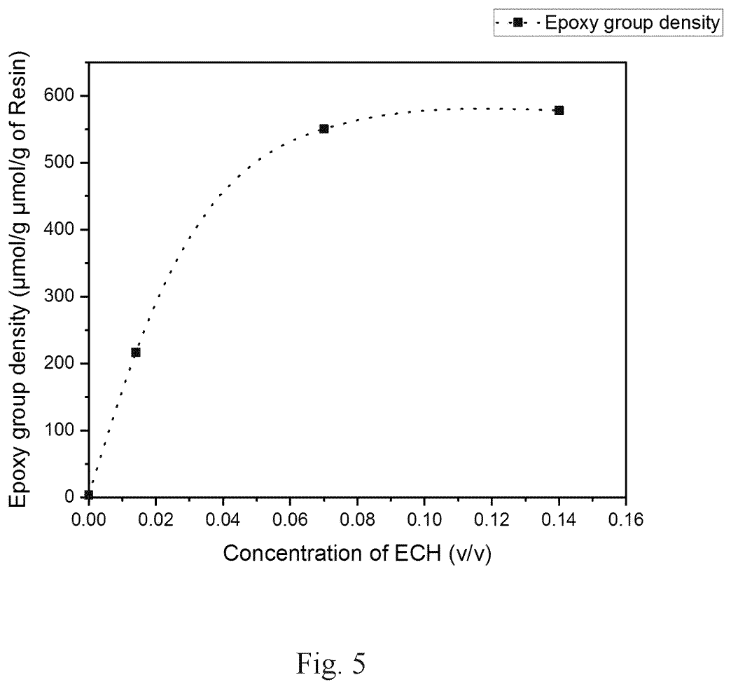

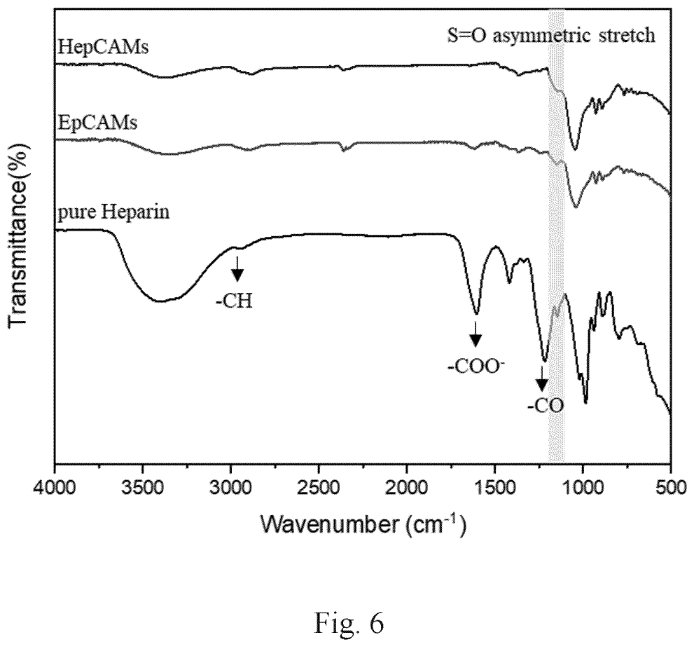

As shown in FIG. 6, the original heparin powder has the following characteristic peaks at —CH stretching at 2941 cm⁻¹, COO-antisymmetric stretching at 1614 cm⁻¹ and —CO asymmetric stretching at 1214 cm⁻¹. The difference in absorption bands between EpCAMs and HepCAMs can be found at the position of 1120 cm⁻¹ to 1208 cm⁻¹, which was due to S═O asymmetric stretching after EpCAMs bonded with heparin. This proves that heparin was successfully covalently bonded to microspheres in the present disclosure.

### (2) Raman Spectroscopy Analysis

In order to confirm whether the surface modification of heparin on agarose microspheres is successful, the present disclosure uses Raman spectroscopy to observe changes in the functional groups and chemical bonds of agarose powder (pure agarose), the EpCAMs of Preparation Example 2 and the HepCAMs of Preparation Example 3.

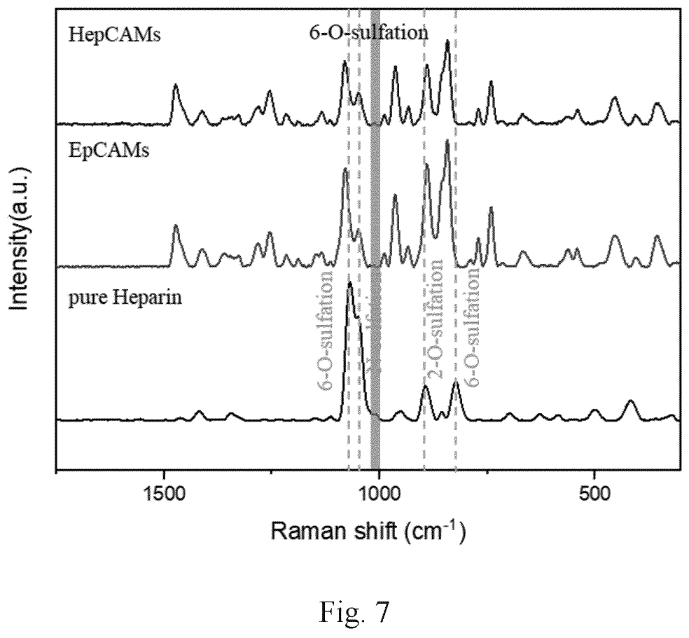

Please refer to FIG. 7, the original heparin powder has the following characteristic peaks: 2-O-sulfation at 892 cm⁻¹, N-sulfation at 1049 cm⁻¹, 821 cm⁻¹, 1008 cm⁻¹ and 1069 cm⁻¹ were both 6-O-sulfation. The difference in the characteristic peak between EpCAMs and HepCAMs can be found at the position of 1008 cm⁻¹, which was due to the 6-O-sulfation after the epoxy group was bonded to heparin. This proves that heparin was successfully covalently bonded to microspheres in the present disclosure.

(3) Content Analysis of EpCAMs Bonded with Heparin

According to Preparation Example 3, the addition amount of heparin was 0.1 wt % to 1.5 wt % of agarose microspheres to perform heparin bonding. Next, toluidine blue (TB) and heparin sodium were combined to form a TB-Heparin complex. Next, n-hexane was added to extract and remove the TB-Heparin complex, and the absorbance of the aqueous phase containing unextracted toluidine blue was measured with a UV spectrophotometer at a wavelength of 631 nm.

Toluidine blue solution preparation: 25 mg TB was dissolved in 500 mL 0.01N hydrochloric acid aqueous solution (containing 0.2 wt % sodium chloride).

Standard solution preparation: heparin solution was prepared with different concentrations (0-25 μg/mL).

Sample preparation: 10 mg of microsphere powder was added into 50 mL of deionized water and mixed evenly.

2 mL of standard solution or sample was added to 3 mL of TB solution and reacted at 37° C. for 2 hours. After the reaction was completed, 3 mL of n-hexane (hexane) was added as the extraction solvent for the toluidine blue-heparin complex, and shaken and mixed thoroughly to stand for layering, and then the extract was removed. The unextracted toluidine blue solution and the clear liquid of the sample were measured by ultraviolet spectrophotometry at a wavelength of 631 nm. The linear relationship between different concentrations of the transmitted standard solution and the absorbance intensity was used as the calibration line to quantify the content of heparin on the surface of the microspheres.

Before discussing different heparin modification concentrations, it is necessary to first understand the absorbance of chemical cross-linked agarose microspheres (CAMs) and surface-modified epoxy group agarose microspheres (EpCAMs) at a wavelength of 631 nm, and then the bonded heparin content was obtained. Next, the results of EpCAMs were the blank control group. The heparin content of CAMs was 4.8 μg/mg of agarose, and the heparin content of EpCAMs was 26.75 μg/mg of agarose. Therefore, the heparin content measured by heparinized microsphere needs to be subtracted from the blank control group (background value: 26.75 μg/mg of agarose).

After subtracting the background value of 26.75 μg/mL of agarose, the effect of heparin concentration (Hep/EpCAMs ratio: 0.25˜1.5) on the bonded heparin content was explored. As shown in Table 3, the content of the bonded heparin increased with the increase of the heparin concentration. However, when the Hep/EpCAMs ratio increased from 1 to 1.5, the change in bonded heparin tends to be flat. It is suggested that insufficient epoxy groups can be effectively modified in the microspheres.

TABLE 3

| | content of heparin |
|---|---|
| Hep/EpCAMs | (μg/mg of agarose) |
| 0.10 | 20 |
| 0.25 | 44.15 |
| 0.50 | 62.2 |
| 0.75 | 70.35 |
| 1.00 | 76.05 |
| 1.50 | 77.11 |

### Example 5 Purification of Exosomes from Cell Culture Fluid

### (1) Exosome Particle Size Distribution and Concentration

In order to observe the particle size distribution and concentration of exosomes after purification of A549 cell culture fluid, the present disclosure uses a nanoparticle tracking analyzer (NTA) for detection.

Size exclusion chromatography: IZON qEV original as the size exclusion chromatography column was used, and the column was washed before use. The concentrated culture medium was added to the column. After the solution had completely flowed through the dispersion plate, PBS was added to the column three times and the liquid was collected to complete the process of purifying extracellular vesicles by size exclusion chromatography.

Affinity capture method: (1) Culture: the concentrated A549 cell culture medium was added to 5 mL microspheres for culture, then the microspheres were centrifuged for precipitation and the supernatant was collected. In order to remove residual proteins and uncaptured extracellular vesicles on the surface of the microspheres, PBS was added to disperse evenly and the microspheres were centrifuged for precipitation again, and the supernatant was collected, the supernatant was the unbonded extracellular vesicles. (2) High-salt elution: 2.15M sodium chloride aqueous solution was added to the above-mentioned cultured microspheres to mix and culture, then the microspheres was centrifuged for precipitation and the supernatant was collected. In order to fully collect the eluted extracellular vesicles, PB was added to disperse evenly and the microspheres was centrifuged for precipitation again, and the supernatant liquid was collected, the supernatant was the captured (bonded) extracellular vesicles.

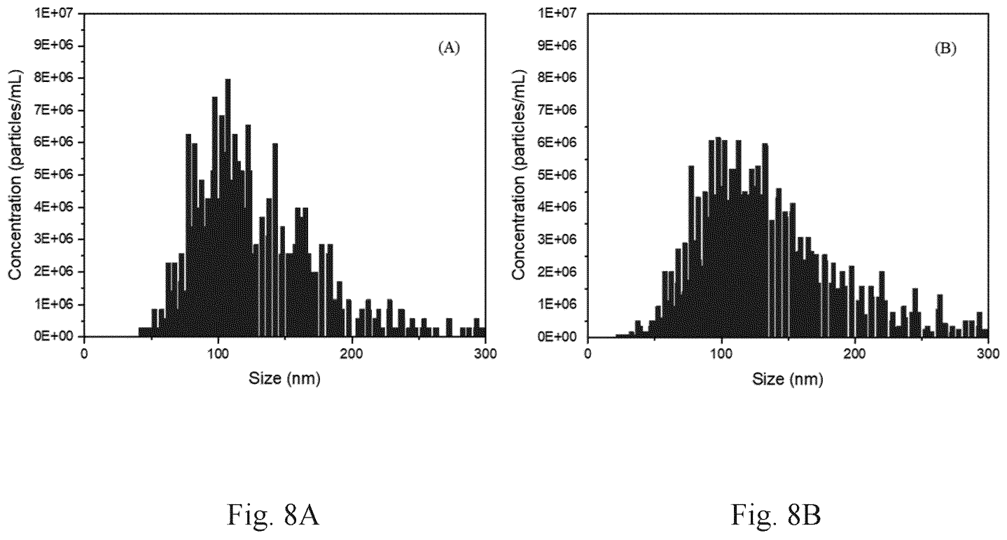

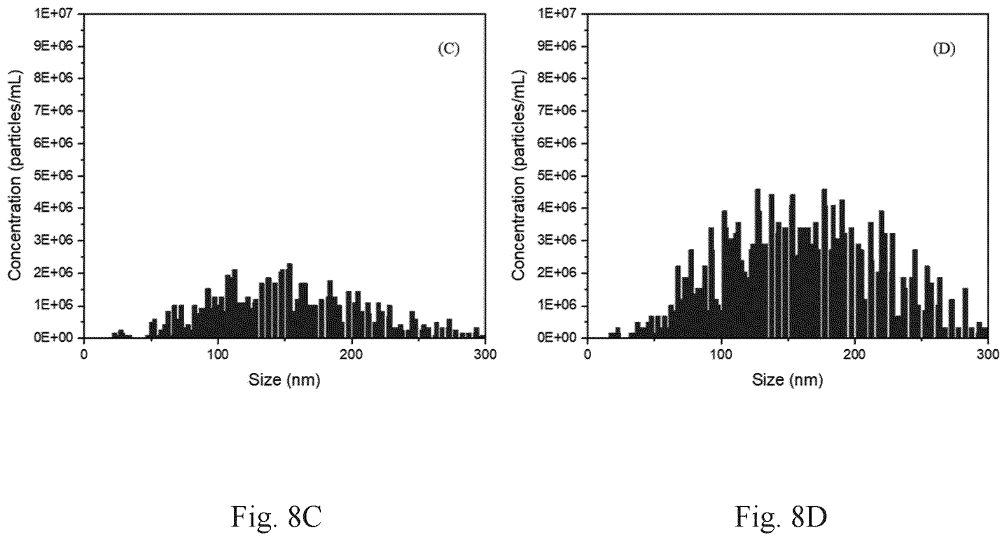

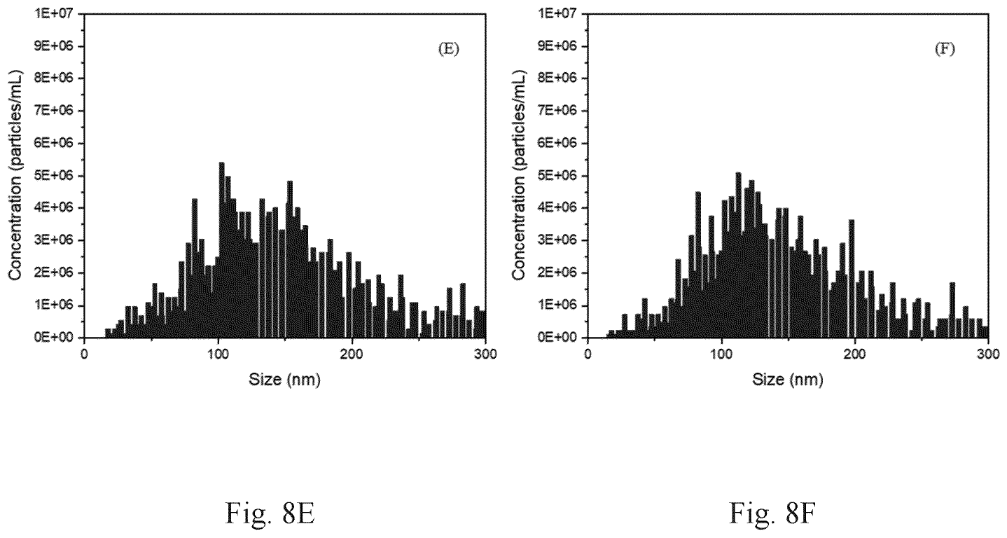

The result of NTA test is as shown in FIG. 8A, and the result is integrated in Table 4. FIG. 8A shows the pre-purified group, which was a sample purified from the cell culture medium through 0.22 μm filtration and 100 kDA concentration centrifuge tube. The particle concentration tested by NTA was 825×10⁹ particles/mL. Except for extracellular vesicles, the pre-purified group also contains many impurities, such as unremoved protein. The pre-purified group had the highest particle concentration among the six groups of samples. FIG. 8B shows that SEC group was a sample of cell culture fluid purified by size exclusion method. Compared with the pre-purified group, the particle concentration dropped sharply from 825×10⁹ particles/mL to 12×10⁹ particles/mL. Comparing the concentration of particles in the exosome-defined size range of 30-150 nm, the SEC group was better than the pre-purified group. It is preliminarily suggested that the vast majority of non-exosome impurities can be removed through the size exclusion method. FIG. 8C shows a sample of cell culture fluid purified by the EpCAMs. A shown in Table 4, it can be observed that the concentration of the EpCAMs particles was the lowest among the six groups. The EpCAMs did not have the heparin affinity function and cannot capture extracellular vesicles. Agarose provided microspheres with extremely low non-specific adsorption capacity and is not easy to adhere to undesirable proteins. FIG. 8D to 8F show modified agarose microspheres with different heparin concentrations (Hep/EpCAM ratios of 0.25, 0.50 and 1.00 wt %). As shown in Table 4, it can be observed that the particle concentration increased with the increase of the heparin concentration, and the median particle size of the HepCAMs group was higher than that of the SEC group.

TABLE 4

| | | Concentration | X₅₀ | |
|---|---|---|---|---|
| | | (10⁹ particles/mL) | Number | Volume |
| Pre-purified group | | 825.00 | 113.3 | 189.0 |
| SEC group | | 12.00 | 119.4 | 206.3 |
| Control group (EpCAMs) | | 0.05 | 141.7 | 713.2 |
| Affinity | 0.25 | 3.00 | 155.7 | 314.1 |
| capture group | 0.50 | 4.65 | 139.1 | 464.2 |
| (HepCAMs) | 1.00 | 5.50 | 133.1 | 514.5 |

### (2) Exosome Purity and Recovery Rate

According to the results of exosome size distribution and concentration, data analysis was conducted for the defined size range of exosomes from 30 nm to 150 nm. The purity and recovery results of different purification methods of exosomes are summarized in Table 5.

From Table 5, it can be observed that the particle concentration of the pre-purification group was 312×10⁹ particles/mL, and the particle concentration obtained after purification by SEC method dropped sharply to 4.38×10⁹ particles/mL. According to the recovery rate formula, the particle recovery rate of the SEC method was 1.40%, which was better than the particle recovery rate of the affinity capture method. However, the protein concentration of the SEC method was 99.83 μg/mL, which was much higher than the protein concentration of the affinity capture method. Therefore, the purity of the SEC method was lower than that of the HepCAMs 0.50 and HepCAMs 1.00 groups. This is due to the fact that although the SEC purification method provided a high-throughput sample processing mode, it cannot perform specific purification and cannot achieve high purity characteristics. On the other hand, the results of the affinity capture method of agarose microspheres show that agarose as a microsphere substrate greatly reduced the effect of non-specific adsorption, and the protein concentration was reduced several times compared to the SEC method. As the heparin concentration increases (Hep/EpCAMs Ratios of 0.25, 0.50 and 1.00 wt %), the concentration of protein adsorbed by microspheres did not change significantly, while the concentration of captured exosome particles showed an increasing trend. Therefore, the purity results of exosomes have been significantly improved; for example, when the Hep/EpCAMs ratio was 1.00 wt %, the purity of exosomes increased by 4.5% compared with the SEC method, and the purity of exosomes increased by 8.8% compared with the epoxidized agarose microspheres.

TABLE 5

| | | Particle | Particle | Total | Purity |
|---|---|---|---|---|---|
| | | (10⁹ parti- | recovery | protein | (10⁷ parti- |
| | | cles/mL) | rate (%) | (μg/mL) | cles/μg) |
| Pre-purified group | | 312 | N/A | 2.5E+6 | N/A |
| SEC group | | 4.38 | 1.40 | 99.83 | 4.38 |
| Control group (EpCAMs) | | 0.01 | 0.004 | 16.02 | 0.08 |
| Affinity | 0.25 | 0.70 | 0.22 | 17.53 | 3.98 |
| capture group | 0.50 | 1.30 | 0.42 | 17.85 | 7.26 |
| (HepCAMs) | 1.00 | 1.62 | 0.52 | 18.19 | 8.90 |

### (3) Exosome Surface Morphology

The present disclosure uses transmission electron microscopy (TEM) to observe the morphology of exosomes, including the results of purifying exosomes using affinity capture method (Affinity) and size exclusion chromatography (SEC). The affinity effect of different heparin concentration microspheres on capturing exosomes based on different capture methods was explored.

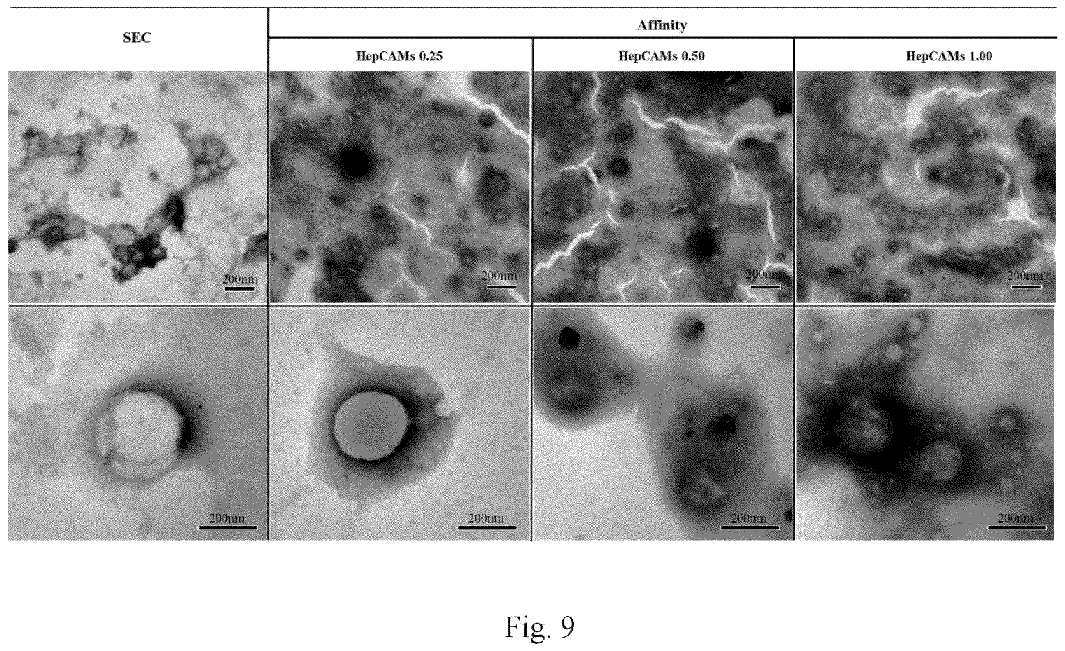

The transmission electron microscope picture in FIG. 9 shows that the purified exosomes can maintain their original integrity and appear as round balls. The size range of the round vesicles in the SEC group was approximately between 70 nm and 150 nm, and the vesicles were surrounded by a large area of aggregated proteins, so that the vesicles were aggregated and were hard to disperse. The size range of the circular vesicles in the Affinity group ranged from approximately 50 nm to 150 nm, and the uniformity of the vesicle dispersion was better than that in the SEC group. As the heparin concentration increased, the size of purified exosomes became smaller and the appearance became more rounded. In the TEM image of HepCAMs 0.50, the concave disc-like structure of typical exosomes was observed, and the vesicle density under the same image area also was increased. The TEM image of HepCAMs 1.00 has the most obvious results that the group with added heparin was more likely to observe exosome aggregation.

### (4) Exosome Immune Characteristics

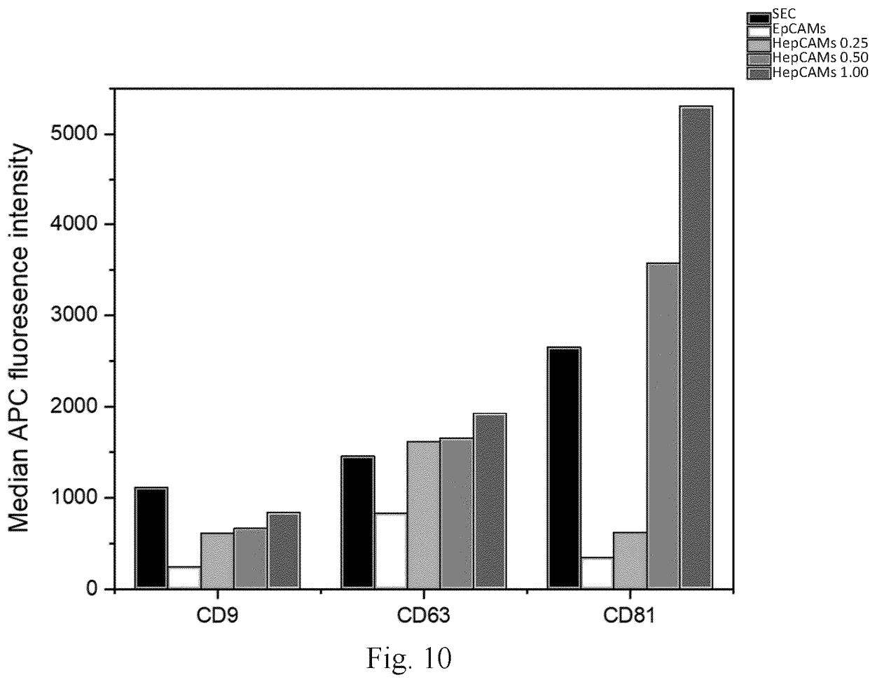

In order to confirm whether samples obtained by different purification methods of exosomes have the exosomes immune characteristics (CD9, CD63 and CD81), the present disclosure combines flow cytometry with MACSPlex EV kit, Human kit to detect the immune characteristics of extracellular vesicles, and the results are summarized in FIG. 10.

As shown in FIG. 10, it can be observed that samples from all groups can display the fluorescence intensity of immune characteristics CD9, CD63 and CD81, indicating that the SEC method or affinity capture method can indeed purify exosomes in cell culture fluid. The fluorescence intensities of CD9, CD63 and CD81 obtained by the SEC method were 1117, 1456 and 2655 respectively. The fluorescence intensity of CD9 was the highest among the five groups, but its expression of CD63 and CD81 was much lower than that of the affinity capture method HepCAMs 0.50 and 1.00 groups. Although the particle concentration obtained by the SEC method was higher, its purity was relatively low, so the expression of the immune characteristics was poor. In addition to not having heparin affinity function, EpCAMs cannot capture extracellular vesicles. Agarose provides microspheres with extremely low non-specific adsorption capacity and is not easy to adhere to undesirable proteins. Therefore, the fluorescence intensity of the immune characteristics was the weakest among the five groups. As the concentration of HepCAMs increased with heparin (Hep/EpCAMs ratio is 0.25, 0.50 and 1.00 wt %), it can be observed that the fluorescence intensity of CD9, CD63 and CD81 increased accordingly. In particular, the CD81 fluorescence intensity of HepCAMs 1.00 group was 5304, which was much higher than that of other groups.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A heparinized microsphere, comprising:
a polymer having at least one functional group, the at least one functional group comprising a hydroxyl group, a carboxyl group, an amino group, or a combination thereof;
heparin; and
a cross-linking agent, the polymer and the heparin cross-linked by the cross-linking agent.

2. The heparinized microsphere of claim 1, wherein the polymer comprises agarose, chitosan, hyaluronic acid, pectin, carboxymethylcellulose, hydroxypropyl methylcellulose, alginate, or a combination thereof.

3. The heparinized microsphere of claim 1, wherein the polymer comprises a physical cross-linked polymer, a chemical cross-linked polymer, or a combination thereof.

4. The heparinized microsphere of claim 1, wherein the cross-linking agent comprises epichlorohydrin.

5. The heparinized microsphere of claim 1, wherein a content of an epoxy group content at a surface of the polymer ranges from 200 μmol/g of the polymer to 600 μmol/g of the polymer.

6. The heparinized microsphere of claim 1, wherein a content of the heparin at a surface of the polymer ranges from 10 μg/mg of the polymer to 90 μg/mg of the polymer.

7. The heparinized microsphere of claim 1, wherein two ends of the cross-linking agent are respectively bonded to the at least one functional group of the polymer and at least one hydroxyl group of the heparin.

8. A method of preparing a heparinized microsphere, comprising:
providing a polymer; and
processing a mixing process with the polymer, heparin and a cross-linking agent, wherein when the polymer and the heparin are in contact with the cross-linking agent, cross-linking is performed to obtain the heparinized microsphere.

9. The method of claim 8, wherein a weight ratio of the polymer to the heparin is from 1:0.1 to 1:2.0.

10. The method of claim 8, wherein the processing the mixing process with the polymer, heparin and the cross-linking agent comprises:
cross-linking the cross-linking agent and the polymer at a volume ratio of from 0.01:1 to 1:1 in an alkaline solution to obtain a chemical cross-linked polymer and resulted swelling ratio (w/w) from 20 to 4; and
mixing the chemical cross-linked polymer, the heparin and the cross-linking agent.

11. The method of claim 8, wherein the providing the polymer comprises:
heating and dissolving a plurality of monomers in water to obtain an aqueous phase mixture;
mixing an oil and a surfactant to obtain an oil phase mixture;
mixing the aqueous phase mixture and the oil phase mixture to obtain a water-in-oil emulsion; and
cooling the water-in-oil emulsion until setting to obtain the polymer.

12. The method of claim 11, wherein the oil comprises alkanes, esters, or a combination thereof.

13. The method of claim 11, wherein the oil comprises petroleum ether, paraffin oil, mineral oil, stearic acid, cottonseed oil, oleyl alcohol, white wax oil or a combination thereof.

14. The method of claim 11, wherein the surfactant comprises sorbitan monooleate (span 80), hydroxylated lanolin, polyoxythylene sorbitol beeswax derivative, propylene glycol fatty acid ester, propylene glycol monolaurate, di(ethylene glycol) monooleate, sodium lauryl ether sulfate (2EO), polyoxythylene sorbitol beeswax derivative, diethylene glycol distearate, or a combination thereof.

15. The method of claim 11, wherein the plurality of monomers comprises agarose, chitosan, hyaluronic acid, pectin, carboxymethylcellulose, hydroxypropyl methylcellulose, alginate, or a combination thereof.

16. The method of claim 8, wherein the processing the mixing process with the polymer, the heparin and the cross-linking agent comprises:
mixing the polymer and the cross-linking agent to obtain a epoxidized microsphere; and
mixing the epoxidized microsphere and the heparin to obtain the heparinized microsphere.

17. The method of claim 16, wherein a volume ratio of the cross-linking agent to the polymer is from 0.001:1 to 0.15:1.

18. The method of claim 16, wherein a weight ratio of the epoxidized microsphere to the heparin is from 1:0.1 to 1:2.0.

19. A method of isolating exosomes, comprising:
mixing a solution containing a plurality of exosomes and a plurality of heparinized microspheres as claimed in claim 1 for combining the plurality of exosomes and the plurality of heparinized microspheres to obtain a plurality of exosome microspheres; and
mixing a salt solution with the plurality of exosome microspheres to isolate the exosomes from the plurality of heparinized microspheres.

20. The method of claim 19, wherein after the mixing the salt solution with the plurality of exosome microspheres, the method further comprises:
mixing the plurality of exosome microspheres and a buffer to obtain a mixed buffer solution; and
centrifuging the mixed buffer solution to obtain a supernatant containing the plurality of exosomes.
